(19) United States
(12) Patent Application Publication
(10) Pub. No.: US 2009/0192596 A1
(43) Pub. Date: Jul. 30, 2009

(54) IMPLANT HAVING A BASE BODY OF A BIOCORRODIBLE ALLOY

(75) Inventor: Nina Adden, Nuernberg (DE)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(73) Assignee: BIOTRONIK VI PATENT AG, Baar (CH)

(21) Appl. No.: 12/362,659

(22) Filed: Jan. 30, 2009

(30) Foreign Application Priority Data
Jan. 30, 2008 (DE) 10 2008 006 654.0

(51) Int. Cl. A61F 2/06; A61F 2/02

(52) U.S. Cl. 623/1.46; 623/11.11

(57) ABSTRACT

An implant having a base body comprised entirely or partially of a biocorrodible metallic material and in which at least the parts of the base body comprising the biocorrodible metallic material are covered entirely or partially with a coating which contains or is comprised of the polymer. At least 90% of the total number of polymer units of the polymer comprise polymer units of formula (1) and polymer units of formula (2)

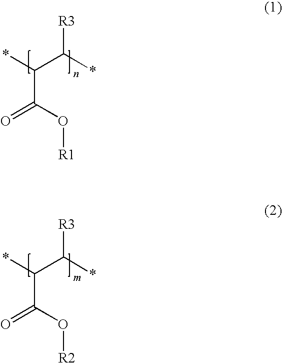

wherein R1 is alkyl, hydroxyalkyl, alkoxyalkyl or cycloalkyl; R2 is a silyl radical that is hydrolyzable in artificial plasma; R3 is hydrogen or methyl, and n+m=10 to 20,000, such that the ratio of n to m is in the range of 1:9 to 9:1.

### PRIORITY CLAIM OR CROSS REFERENCE TO RELATED APPLICATIONS

This patent application claims priority to German Patent Application No. 10 2008 006 654.0, filed Jan. 30, 2008, the disclosure of which is incorporated herein by reference in its entirety.

### FIELD

The present disclosure relates to an implant having a base body which is comprised entirely or in part of a biocorrodible metallic material and in which at least the parts of the base body comprised of the biocorrodible metallic material are covered partially or completely by a coating which is, in turn, comprised of or contains a polymer.

### BACKGROUND

Implants are used in a variety of ways in modern medical technology. Among other things, implants are used for supporting blood vessels, hollow organs and duct systems (endovascular implants), for fastening and temporary fixation of tissue implants and tissue transplants, and also for orthopedic purposes, e.g., as nails, plates or screws.

For example, implantation of stents has become established as one of the most effective therapeutic measures for treatment of vascular diseases. The purpose of stents is to assume a supporting function in a patient's hollow organs. Stents of a traditional design, therefore, have a filigree supporting structure of metallic struts which are initially in a compressed form for introduction into the body and then are dilated at the site of application. One of the main areas of application of such stents is for permanently or temporarily dilating vasoconstrictions, in particular, constrictions (stenoses) of the coronary vessels, and maintaining vascular patency. In addition, there are also known aneurysm stents that serve to support damaged vascular walls.

The base body of each implant, in particular, a stent, comprises an implant material. For purposes of the present disclosure, an implant material means a nonviable material that is used for an application in medicine and interacts with biological systems. The basic prerequisites for use of a material as an implant material which is in contact with the biological environment when used as intended is its biological compatibility (biocompatibility). For purposes of the present disclosure, biocompatibility means the ability of a material to induce an appropriate tissue reaction in a specific application. This includes adaptation of the chemical, physical, biological and morphological surface properties of an implant to the recipient tissue with the goal of a clinically desired interaction. The biocompatibility of the implant material also depends on the course of the reaction of the biosystem into which the implant is implanted over time. Irritation and inflammation occur in the relatively short term, possibly leading to tissue changes. Biological systems thus react in different ways depending on the properties of the implant material. Implant materials can be subdivided into bioactive, bioinert and degradable/absorbable materials, depending on the reaction of the biosystem. For the purposes of the present disclosure, only degradable/absorbable, metallic implant materials are of interest. Degradable/absorbable, metallic implants are also referred to below as biocorrodible metallic materials.

The use of biocorrodible metallic materials is recommended, in particular, because in most cases the implant need only remain in the body for a short period of time to fulfill the medical purpose. Implants of permanent materials, i.e., materials that are not degraded in the body, must be removed because rejection reactions may occur in the body in the medium range and in the long range, even if there is a high biocompatibility.

One approach to avoid another surgical procedure is thus comprised of making the implant entirely or in part of a biocorrodible metallic material. For purposes of the present disclosure, biocorrosion means microbial processes or processes due simply to the presence of endogenous media, leading to a gradual degradation of the structure comprised of the material. At a certain point, the implant, or at least the part of the implant comprised of the biocorrodible material, loses its mechanical integrity. The degradation products are largely absorbed by the body. In the best case, the degradation products, such as magnesium, for example, may even have a positive therapeutic effect on the surrounding tissue. Small quantities of unabsorbable alloy constituents can be tolerated.

Known biocorrodible metallic materials include pure iron and biocorrodible alloys of the main elements magnesium, iron, zinc, molybdenum and tungsten. German Patent Application No. 197 31 021 discloses that medical implants should be comprised of a metallic material whose main constituent is an element from the group consisting of alkali metals, alkaline earth metals, iron, zinc and aluminum. Alloys based on magnesium, iron and zinc have been described as especially suitable. Secondary constituents of the alloys may be manganese, cobalt, nickel, chromium, copper, cadmium, lead, tin, thorium, zirconium, silver, gold, palladium, platinum, silicon, calcium, lithium, aluminum, zinc and iron. In addition, German Patent Application No. 102 53 634 describes the use of a biocorrodible magnesium alloy containing >90% magnesium, 3.7-5.5% yttrium, 1.5-4.4% rare earth metals and remainder <1%. Such alloys are suitable, in particular, for production of an endoprosthesis, e.g., in the form of a stent. Regardless of the advances made in the field of biocorrodible metal alloys, the alloys known so far can be used only to a limited extent because of their corrosion behavior. In particular, the relatively rapid biocorrosion of magnesium alloys limits the scope of their use.

Traditional technical applications of molded bodies comprised of metallic materials, in particular, magnesium alloys, outside of medical technology usually require extensive suppression of corrosion processes. Accordingly, the goal of most technical methods for improving corrosion behavior is to completely inhibit corrosion processes. However, the goal to improve corrosion behavior of the biocorrodible metallic materials in the present disclosure should lie not in complete suppression but instead only inhibition of corrosive processes. For this reason alone, most of the known measures for improving corrosion protection are unsuitable. Furthermore, for a medical technical use, toxicological aspects must also be taken into account. In addition, corrosive processes depend greatly on the medium in which the corrosive processes take place and, therefore, transferability of findings about corrosion prevention obtained under traditional environmental conditions in a technical field should not be applicable to an unlimited extent to the processes taking place in a physiological environment.

One approach of known technical methods for improving corrosion behavior (in the sense of increasing corrosion protection) provides for a corrosion-preventing layer to be produced on the molded body comprised of the metallic material. Known methods for creating a corrosion-preventing layer have been developed and optimized from the standpoint of technical use of the coated molded body, but not for medical technical use in biocorrodible implants in a physiological environment. These known methods include, for example, application of polymers or inorganic top coats, creating an enamel, chemical conversion of the surface, hot gas oxidation, anodization, plasma sputtering, laser beam fusion, PVD methods, ion implantation or lacquering.

One aspect of the present disclosure provides an improved or at least an alternative coating for an implant comprised of biocorrodible metallic material that produces a temporary inhibition but not complete suppression of corrosion of the material in a physiological environment.

### SUMMARY

The present disclosure describes several exemplary embodiments of the present invention.

One aspect of the present disclosure provides an implant comprising a base body comprising at least partially a biocorrodible metallic material, wherein at least the parts of the base body comprised of the biocorrodible metallic material are covered at least partially with a coating containing a polymer, wherein at least 90% of the total number of polymer units in the polymer comprises polymer units of formula (1) and polymer units of formula (2):

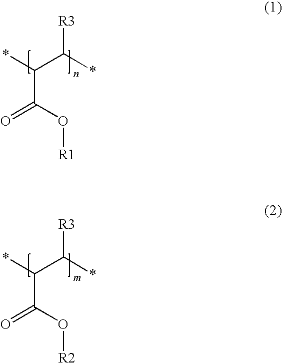

where R1 is selected from the group consisting of either alkyl, hydroxyalkyl, alkoxyalkyl and cycloalkyl; R2 is a silyl radical hydrolyzable in artificial plasma; R3 is either hydrogen or methyl; and n+m=10 to 20,000, such that the ratio of n to m is in the range of 1:9 to 9:1.

The present disclosure is based on the finding that polymers based on the aforementioned esters of acrylic acid/methacrylic acid have a predominantly hydrophobic behavior and accordingly have a low solubility in aqueous media, in particular, synthetic plasma. If these polymers are thus used as a coating material for at least the parts of an implant that are comprised of a biocorrodible metallic material, then this material is first protected from corrosive degradation by the coating. However, hydrolysis of the silyl esters of the polymer units of formula (2) takes place gradually in the synthetic medium. Associated with this, the hydrophilic character of the polymer increases and its solubility in the artificial plasma also increases. In other words, with progressive hydrolysis of the polymer units of formula (2), the solubility of the polymer is increased and, as soon as the degradation has advanced to a sufficient extent, the biocorrodible metallic material underneath is degraded.

An important difference in comparison with coatings comprised of known biocorrodible polymers is that the hydrolysis of the silyl esters described herein does not yield the monomers as a degradation product but instead the basic chain structure of the polymer is retained. This has the advantage that a high burden of low-molecular degradation products on the tissue in comparison with traditional biocorrodible polymers can be avoided. Precisely these low-molecular degradation products are suspected of being the starting point for adverse reactions in the patient's body which can have a negative influence on the course of healing or, in the worst case, may even prevent healing. The soluble polymers obtained by hydrolysis of the silyl esters, however, are largely excreted without any negative interactions.

### DETAILED DESCRIPTION

The polymer contains the polymer units of formulas (1) and (2),

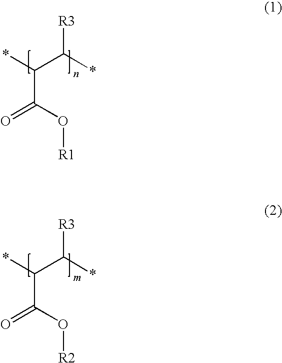

thus being a copolymer that contains one species each as a polymer unit that falls under the formulas (1) and (2). It is thus also conceivable and contemplated as part of the present disclosure that the polymer contains different species subsumed under the polymer units of formulas (1) or (2). A distribution of the polymer units in the polymer is of subordinate importance for the purposes of the present disclosure so that, as a rule, block copolymers or graft copolymers may also be used. For purposes of the present disclosure, the term copolymer is a general designation for polymers comprising two or more different types of monomers and produced by joint copolymerization. The polymer may also contain up to 10% polymer units formed by free-radical polymerization of a vinyl monomer different from formulas (1) and (2). For example, this additional polymer unit may be vinyl pyrrolidone or vinyl acetate. In this way, the solubility behavior of the polymer may additionally be influenced, for example.

Artificial blood plasma, as specified for biocorrosion investigations according to published standard EN ISO 10993-15:2000 (composition NaCl 6.8 g/L, CaCl₂ 0.2 g/L, KCl 0.4 g/L, MgSO₄ 0.1 g/L, NaHCO₃ 2.2 g/L, Na₂HPO₄ 0.126 g/L, NaH₂PO₄ 0.026 g/L) is used as the medium for testing the hydrolyzability of silyl esters and/or the solubility behavior of polymers. A sample of the polymer to be investigated is stored in a defined amount of test medium at 37° C. in a sealed sample container. At intervals of time from a few hours up to several months, depending on the anticipated degradation behavior of the silyl ester, the extent of hydrolytic degradation is determined on the samples or by testing the artificial medium in a way known to those skilled in the art. The artificial blood plasma according to EN ISO 10993-15:2000 corresponds to a blood-like medium and thus simulates a physiological environment.

According to one exemplary embodiment, the polymer has a solubility lower than 0.01 g/L in artificial blood plasma after 24 hours at a temperature of 37° C. based on definition of the substituents R1 through R3 and the definition of n and m. In addition, or as an alternative, by a stipulation of the substituents R1 to R3 and by definition of n and m, the polymer has a solubility in artificial blood plasma in the range of 0.2 to 0.5 g/L after 30 days at a temperature of 37° C. in this plasma. The stipulations mentioned hereinabove for the solubility after 24 hours and/or 30 days should ensure that degradation of the biodegradable metallic constituents of the implant beneath the polymer coating does not begin immediately after implantation but instead is inhibited. After 30 days, however, there should be a significant transport of polymer away from the implant as a result of the increase in solubility which then occurs. The latter period of time is based on the fact that functionality of the constituents from the biodegradable metallic material must be maintained only for two to three months for many implants. This constituent of the implant should then be degraded promptly thereafter. The solubility of the polymer after 30 days must, therefore, be great enough to maintain the time frame noted hereinabove. To achieve the stated solubility behavior, the following process may be used:

Starting from a concrete polymer obtainable, e.g., by polymerization of methyl methacrylate and trimethylsilyl methacrylate, the solubility of this polymer in artificial blood plasma is determined after 24 hours and/or 30 days at a temperature of 37° C. If the solubility does not have the properties desired for the specific application, the practitioner can influence the solubility through the following variations:
(1) Instead of methyl as the radical R1 of the polymer unit of formula (1), a long-chain alkyl radical or a cycloalkyl radical is selected if the hydrophobicity of the polymer material is to be increased. Conversely, a more hydrophilic radical, i.e., a hydroxyalkyl radical or a short-chain alkoxylalkyl radical, is selected when the hydrophilicity and thus the solubility are to be increased.
(2) The hydrolysis rate of the silyl esters usually increases with an increase in the size of the substituents on the silicon. Thus, through the choice of the silyl radical, which hydrolyzes relatively slowly, reaching the required solubility for transporting the polymer away from the implant is delayed over time.
(3) The solubility of the polymer in artificial blood plasma naturally also depends on the number of hydrophilic groups which are formed mainly by hydrolysis of the silyl esters. Thus, if the amount of silyl esters, i.e., the polymer units of formula (2) in the polymer is increased, then the completely hydrolyzed polymer will have a greatly increased solubility in comparison with that of a polymer having fewer silyl radicals.
(4) In addition, the solubility also depends to a lesser extent on the molecular weight of the polymer. As a rule, an increase in molecular weight is associated with a negative effect on solubility in artificial blood plasma.

Those skilled in the art thus have available the four parameters described hereinabove, the influence of which practitioners tend to know and which practitioners can vary independently of one another to arrive at a product having the properties desired for this specific application.

R1 is preferably an unsubstituted, hydroxy-substituted or C1-C10 alkoxy-substituted C1-C10 alkyl radical or a C2-C10 cycloalkyl radical.

R1 is especially preferably methyl, ethyl, propyl, 2-methylethyl, butyl, 2-methylpropyl, 2,3-dimethylethyl, cyclohexyl, 2-hydroxyethyl, 3-hydroxypropyl or 2-methoxyethyl. R1 is most especially preferably methyl, ethyl, propyl, or butyl. R1 is methyl, in particular.

With the exemplary embodiments noted hereinabove for the substituents R1, in particular, it is also preferable for R2 to be trimethylsilyl, triethylsilyl, tripropylsilyl or tributylsilyl. More preferably, R2 is trimethylsilyl or triethylsilyl,.

Furthermore, it is preferable if the ratio of n to m is in the range of 1:2.33 to 1.5:1.

Furthermore, an exemplary embodiment in which the polymer unit of formula (1) is methyl methacrylate or methyl acrylate, and the polymer unit of formula (2) is trimethylsilyl methacrylate is especially preferred.

Furthermore, according to other exemplary embodiments, the average molecular weight of the polymers is in the range of 1000 g/mol to 1,000,000 g/mol, especially preferably in the range of 5000 to 500,000 g/mol, and still more preferably, 40,000 to 200,000 g/mol.

Additives may be added to the polymer coating to facilitate processing, for example. It is also conceivable for the coating to be used as a matrix for active ingredients that are released into the surrounding tissue after implantation.

The polymers can be produced by free-radical polymerization in a known manner, e.g., by analogy with the procedure of P. Durand et al. (disclosed in POLYMER, vol. 35, 1994, pages 4392 to 4396). The molecular weight can be influenced via the concentration of the initiator for the free-radical polymerization, among other routes.

The biocorrodible metallic material is preferably pure iron or a biocorrodible alloy selected from the group of elements consisting of magnesium, iron, zinc, molybdenum and tungsten. The material is a biocorrodible magnesium alloy, in particular. For purposes of the present disclosure, the term “alloy” means primarily a metallic structure having as its main component magnesium, iron, zinc, molybdenum or tungsten. The main component is the alloy component that is present in the alloy in the largest amount by weight. The amount of the main component is preferably greater than 50 wt %, in particular, greater than 70 wt %.

Especially preferred is a magnesium alloy having the following composition: 5.2-9.9 wt % rare earth metals, including 3.7-5.5 wt % yttrium and remainder <1 wt %, where magnesium accounts for the remainder of the alloy up to 100 wt %. This magnesium alloy has already confirmed its special suitability in clinical trials, i.e., the magnesium alloy has a high biocompatibility, favorable processing properties, good mechanical characteristics and adequate corrosion behavior for the intended purposes. For purposes of the present disclosure, the term “rare earth metals” means scandium (21), yttrium (39), lanthanum (57) and the 14 elements that follow lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70) and lutetium (71). In addition, magnesium alloys containing up to 6 wt % zinc are preferred. An especially preferred magnesium alloy has the composition 0.5-10 wt % yttrium, 0.5-6 wt % zinc, 0.05-1 wt % calcium, 0-0.5 wt % manganese, 0-1 wt % silver, 0-1 wt % cerium as well as 0-1 wt % zirconium or 0-0.4 wt % silicon, where the amounts are based on percent by weight of the alloy, and magnesium and the manufacturing-related impurities account for the remainder of the alloy up to 100 wt %.

The alloys of the elements magnesium, iron, zinc, molybdenum or tungsten are to be selected with regard to their composition so that they are biocorrodible. For purposes of the present disclosure, the term “biocorrodible” means alloys in which a degradation/rearrangement takes place in a physiological environment so that the part of the implant comprising the material is entirely or at least predominantly no longer present. The composition of the alloy is thus to be selected so that the alloy is biocorrodible.

Artificial blood plasma such as that stipulated according to published standard EN ISO 10993-15:2000 for biocorrosion tests (composition: NaCl 6.8 g/L, CaCl₂ 0.2 g/L, KCl 0.4 g/L, MgSO₄ 0.1 g/L, NaHCO₃ 2.2 g/L, Na₂HPO₄ 0.126 g/L, NaH₂PO₄ 0.026 g/L) is used as the test medium for testing the corrosion behavior of an alloy in question. A sample of the alloy to be tested is stored in a defined amount of test medium at 37° C. in a sealed test container. At intervals of time (based on the anticipated corrosion behavior) from a few hours up to several months, the samples are then removed and tested for traces of corrosion by known methods. The artificial blood plasma according to EN ISO 10993-15:2000 corresponds to a blood-like medium and thus simulates a physiological environment.

For purposes of the present disclosure, the term “corrosion” refers primarily to the reaction of a metallic material with its environment whereby a measurable change in the material is induced leading to an impairment of the function of the component when the material is used in a component. A corrosion system is comprised primarily of the corroding metallic material and a liquid corrosion medium which simulates in its composition the conditions in a physiological environment or a physiological medium, in particular, blood. With regard to the materials, corrosion is influenced by factors such as the composition and pretreatment of the alloy, microscopic and submicroscopic inhomogeneities, boundaries on properties, temperature and mechanical stress state and, in particular, the composition of a layer covering the surface. With regard to the medium, the corrosion process is influenced by conductivity, temperature, temperature gradients, acidity, volume/surface ratio, concentration difference and flow rate.

Redox reactions take place at the phase boundary between the material and the medium. For a protective and/or inhibiting effect, protective layers that are present and/or the products of the redox reactions must develop a sufficiently dense structure against the corrosion medium, must have an increased thermodynamic stability based on the environment, and must have little or no solubility in the corrosion medium. At the phase boundary, or more specifically in a double layer that develops in this area, adsorption and desorption processes take place. The processes taking place in the double layer are characterized by cathodic, anodic and chemical subprocesses taking place there. Deposits of foreign substances, contaminants and corrosion products influence the corrosion process. The processes involved in corrosion are thus extremely complex and cannot be predicted or can be predicted only to a limited extent especially in conjunction with a physiological corrosion medium, i.e., blood or artificial plasma, because of a lack of reference data. For this reason alone, discovering a corrosion-inhibiting coating, i.e., a coating that serves to only temporarily reduce the corrosion rate of a metallic material of the composition defined above in a physiological medium, is a measure outside of the routine practice of those skilled in the art.

The corrosion process can be quantified by stating a corrosion rate. Prompt degradation is associated with a high corrosion rate and vice versa. Based on the degradation of the entire solid body, a surface modified according to the present disclosure will lead to a reduction in the corrosion rate. In the case of coronary stents, the mechanical integrity of the structure should preferably be maintained for a period of three months after implantation.

For purposes of the present disclosure, implants are devices introduced into the body by a surgical procedure and include fastening elements for bones, e.g., screws, plates or nails, surgical suture materials, intestinal clamps, vascular clips, prostheses in the area of hard tissue and soft tissue and anchoring elements for electrodes, in particular, for pacemakers or defibrillators. The implant is comprised entirely or in part of the biocorrodible material. When the implant is comprised of the biocorrodible material only in part, the implant must be coated accordingly.

The implant is preferably a stent. Stents of the traditional design have a filigree structure of metallic struts which are present initially in an unexpanded state for introduction into the body and are then widened into an expanded state at the site of application. In the case of stents, there are special requirements of the corrosion-inhibiting layer. The mechanical load and the material during expansion of the implant have an influence on the course of the corrosion process, and it is assumed that stress corrosion cracking in the areas under stress is increased. A corrosion-inhibiting layer should take this into account. In addition, a hard corrosion-inhibiting layer might rupture during expansion of the stent and then cracking of the layer during expansion of the implant might be unavoidable. Finally, the dimensions of the filigree metallic structure should be taken into account and, if possible, only a thin but uniform corrosion-inhibiting layer should be produced. It has been found that application of the coating according to the present disclosure meets these requirements entirely or at least in part.

The present disclosure is explained in greater detail below on the basis of an exemplary embodiment.

### Production and Choice of the Polymers:

The monomers trimethylsilyl methacrylate TMSM (98%, Aldrich) and methyl methacrylate MMA (99%, Aldrich) are purified before polymerization according to Perrin, if necessary (Perrin, Purification of Laboratory Chemicals). Azobisisobutyronitrile (AIBN) is recrystallized in methanol before being used.

Various ratios of the monomers can be tested as follows: 1-molar solutions of the monomers are prepared in dry toluene and a stock solution (1 molar) of AIBN in toluene is prepared. Dry screw-top test tubes are flushed with nitrogen and filled with the respective amount of monomer solution (see Table 1). Polymerization is initiated by adding the initiator solution and raising the temperature. After approximately 15 hours, polymerization is terminated and the polymer is precipitated in petroleum ether and separated. The polymers are analyzed after re-precipitating twice from dry THF in petroleum ether.

TABLE 1

| | V (TMSM) | V (MMA) | V (AIBN] |
|---|---|---|---|
| | [mL] | [mL] | [μL] |
| A | 1 | 9 | 100 |
| B | 2 | 8 | 100 |
| C | 3 | 7 | 100 |
| D | 4 | 6 | 100 |
| E | 5 | 5 | 100 |
| F | 6 | 4 | 100 |
| G | 7 | 3 | 100 |
| H | 8 | 2 | 100 |
| I | 9 | 1 | 100 |

Of the polymers, films with a weight of approximately 100 mg each should be prepared. They are stored in artificial plasma at 37° C. The films are rinsed and dried after 1, 8, 16, 30 and 90 days, for example, and the weight loss is determined.

### Coating:

Ten stents each of the commercially available magnesium alloy WE43 (designation according to ASTM) which have a rare earth metal content of approximately 3 wt % not including yttrium and an yttrium content of approximately 4 wt % are coated with polymers A to I. The coating weight should amount to 400 μg per stent. The layer thickness should be approximately 4 μg.

### Degradation Tests:

The coated stents should be stored in artificial plasma for 14 days. Then the percentage degradation of the stents can be evaluated.

All patents, patent applications and publications referred to herein are incorporated by reference in their entirety.

What is claimed is:

1. An implant, comprising:
a base body comprising at least partially a biocorrodible metallic material, wherein at least the parts of the base body comprised of the biocorrodible metallic material are covered at least partially with a coating containing a polymer,
wherein at least 90% of the total number of polymer units in the polymer comprises polymer units of formula (1) and polymer units of formula (2): 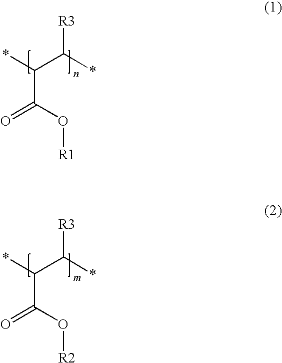
where R1 is selected from the group consisting of alkyl, hydroxyalkyl, alkoxyalkyl and cycloalkyl;
R2 is a silyl radical hydrolyzable in artificial plasma;
R3 is either hydrogen or methyl; and
n+m=10 to 20,000, such that the ratio of n to m is in the range of 1:9 to 9:1.

2. The implant of claim 1, wherein the polymer has a solubility lower than 0.01 g/L after 24 hours in artificial plasma at a temperature of 37° C. based on the choice of the substituents R1 to R3 and the definition of n and m.

3. The implant of claim 1, wherein the polymer has a solubility in the range of 0.2 to 0.5 g/L after 30 days in artificial plasma at a temperature of 37° C., based on a choice of substituents R1 to R3 and the determination of m and n.

4. The implant of claim 1, wherein RI is selected from the group consisting of an unsubstituted C1-C10 alkyl radical, hydroxy-substituted C1-C10 alkyl radical, C1-C10 alkoxy-substituted C1-C10 alkyl radical and a C2-C10 cycloalkyl radical.

5. The implant of claim 1, wherein R1 is selected from the group consisting of methyl, ethyl, propyl, 2-methylethyl, butyl, 2-methylpropyl, 2,3-dimethylethyl, cyclohexyl, 2-hydroxyethyl, 3-hydroxypropyl and 2-methoxyethyl.

6. The implant of claim 1, wherein R1 is selected from the group consisting of methyl, ethyl, propyl, and butyl.

7. The implant of claim 1, wherein R1 is methyl.

8. The implant of claim 1, wherein R2 is selected from the group consisting of trimethylsilyl, triethylsilyl, tripropylsilyl and tributylsilyl.

9. The implant of claim 1, wherein R2 is selected from the group consisting of trimethylsilyl and triethylsilyl.

10. The implant of claim 1, wherein the ratio of n to m is in the range of 1:2.33 to 1.5:1.

11. The implant of claim 1, wherein the polymer unit of formula (1) is either methyl methacrylate or methyl acrylate and the polymer unit of formula (2) is trimethylsilyl methacrylate.

12. The implant of claim 1, wherein the average molecular weight of the polymers is in the range of 1000 g/mol to 1,000,000 g/mol.

13. The implant of claim 1, wherein the average molecular weight of the polymers is in the range of 5000 to 500,000 g/mol.

14. The implant of claim 1, wherein the average molecular weight of the polymers is in the range of 40,000 to 200,000 g/mol.

15. The implant of claim 1, wherein the biocorrodible metallic material is either pure iron or a biocorrodible alloy selected from the group consisting of magnesium, iron, zinc, molybdenum and tungsten.

16. The implant of claim 1, wherein the biocorrodible metallic material is a magnesium alloy.

17. The implant of claim 1, wherein the implant is a stent.

18. The implant of claim 1, wherein the polymer comprises up to 10% polymer units formed by free-radical polymerization of a vinyl monomer differing from formulas (1) and (2).
